(19) United States
(12) Patent Application Publication
(10) Pub. No.: US 2020/0359916 A1
(43) Pub. Date: Nov. 19, 2020

(54) BLOOD PRESSURE METER AND METHOD FOR MEASURING BLOOD PRESSURE USING THE SAME

(71) Applicant: CHARMCARE CO., LTD., Seoul (KR)

(72) Inventor: Dong Hwa LEE, Yongin-si, Gyeonggi-do (KR)

(73) Assignee: CHARMCARE CO., LTD., Seoul (KR)

(21) Appl. No.: 16/765,868

(22) PCT Filed: Nov. 19, 2018
(86) PCT No.: PCT/KR2018/014228

§ 371 Date: May 21, 2020

(30) Foreign Application Priority Data
Nov. 23, 2017 (KR) 10-2017-0157115

(51) Int. Cl. A61B 5/021; A61B 5/024; A61B 5/00

(52) U.S. Cl. CPC A61B 5/02116; A61B 5/02416; A61B 2562/0247; A61B 2562/0219; A61B 5/7278

(57) ABSTRACT

Disclosed are an optical measurement type blood pressure meter and a method for measuring blood pressure. The blood pressure meter according to an aspect of the present invention comprises: a pulse wave measuring unit for measuring arterial pulse waves; a blood pressure difference calculating unit for calculating a difference between blood pressure values generated by a height difference between two certain points where the pulse waves are measured; and a blood pressure wave calculating unit for converting the pulse waves measured at the two points into blood pressure waves by using the difference between the blood pressure values. The blood pressure meter according to an aspect of the present invention may be provided as a wearable blood pressure meter, that is, a wrist blood pressure meter or a finger blood pressure meter that can be worn on a predetermined part of the human body, such as the wrist or the finger. In addition, the present invention can be a blood pressure meter for measuring blood pressure by allowing the finger to be in contact with an optical measuring sensor of a smart phone. The present invention enables the blood pressure meter for measuring blood pressure by using pulse waves to reflect, in a blood pressure calculation, rapid changes in a blood vessel state which largely influence a blood pressure measurement in addition to a blood flow rate, thereby enabling blood pressure to be measured with improved accuracy.

### TECHNICAL FIELD

The present invention relates to a blood pressure meter and a method for measuring blood pressure, and more particularly, to a blood pressure meter of converting arterial pulse waves into waveforms (blood pressure waves) of blood pressure to obtain the blood pressure.

### BACKGROUND ART

In general, measuring pressure that blood has an effect on the walls of blood vessels is called blood pressure, and the heart repeats contraction and relaxation about 60 to 80 times per minute. When the heart contracts and pushes out blood, the pressure on the blood vessels is called ‘systolic blood pressure’, which is called ‘maximal blood pressure’ because the pressure is the highest. In addition, pressure of blood vessels when the heart relaxes and receives the blood is called ‘diastolic blood pressure, which is called ‘minimal blood pressure’ because the pressure is the lowest.

Generally, the blood pressure of a normal person has 120 mmHg of systolic blood pressure and 80 mmHg of diastolic blood pressure. At least one of 4 adults in Korea has hypertension and since the age of 40, the rate has been increased rapidly, while there are also patients classified as hypotension.

The hypertension is a problem because the hypertension may cause other complications that may threaten life, such as eye disease, kidney disease, arterial disease, brain disease, and heart disease, if the hypertension is left without proper management. Accordingly, in patients with or at risk of complications, continuous measurement and management of blood pressure should be performed.

As the interest in health and diseases related to adult diseases such as hypertension described above increases, various types of blood pressure measuring devices have been developed. The blood pressure measuring types include a Korotkoff sounds type, an oscillometric type, a tonometric type, and the like.

The Korotkoff sounds type is a typical pressure measuring type and a method of measuring pressure at the moment when pulse sound is first heard as systolic pressure and measuring pressure at the moment when the pulse sound disappears as diastolic pressure, in a process of blocking and then decompressing the flow of blood by applying sufficient pressure to a body part through which arterial blood passes.

In addition, the oscillometric type and the tonometric type are types to be applied to a digitized blood pressure measuring device. Like the Korotkoff sounds type, the oscillometric type measures the systolic pressure and the diastolic pressure by sensing pulse waves generated in a process of sufficiently pressing a body part through which arterial blood passes and then decompressing the body part at a constant rate so that the arterial blood flow is blocked, or a process of pressing the body part to increase the pressure at a constant rate.

Here, pressure at a certain level may be measured as systolic blood pressure or diastolic blood pressure compared to the moment when the amplitude of the pulse wave is maximum, and pressure when a change rate of the pulse wave amplitude is rapidly changed may also be measured as systolic blood pressure or diastolic blood pressure.

In addition, in the process of decompressing the blood pressure at a constant rate after pressing, the systolic blood pressure is measured earlier than the moment when the amplitude of the pulse wave is maximum and the diastolic blood pressure is measured later than the moment when the amplitude of the pulse wave is maximum. On the contrary, in the process of increasing the blood pressure at a constant rate, the systolic blood pressure is measured later than the moment when the amplitude of the pulse wave is maximum and the diastolic blood pressure is measured earlier than the moment when the amplitude of the pulse wave is maximum.

The tonometric type is a type of applying to the body part constant pressure having a magnitude which does not completely block the arterial blood flow and measuring the blood pressure continuously using a magnitude and a form of pulse waves generated at this time.

The apparatus for measuring blood pressure in various types described above, that is, the blood pressure meter is the most basic medical equipment for measuring blood pressure, which is the basis of a health index, and is not only provided almost necessarily in general hospitals, but also has been frequently used for measuring personal blood pressure even in homes and sports centers.

Most blood pressure meters currently in use are designed to measure at the upper arm similar to the height of the heart, but products capable of measuring blood pressure on the wrist or finger have been developed for convenience in carrying and measuring. The wrist blood pressure meter or finger blood pressure meter is smaller in size than the upper arm blood pressure meter to have an advantage of being convenient to be carried and easy to be measured at any time.

Meanwhile, when blood pressure is measured by using pulse waves, for example, when blood pressure is measured by using optical arterial waves (pulse waves measured by a photo-plethysmography (PPG)), instability according to blood vessel conditions may be caused.

Accordingly, the present inventors have developed a blood pressure meter and a method for acquiring blood pressure capable of reflecting a rapid change in a blood vessel state having a large effect on blood pressure together with a blood flow rate, for example, a cross-sectional change in a blood vessel in a blood pressure calculation.

In this regard, in Korean Patent Publication No. 10-2010-0118331 as a prior art, there are disclosed an apparatus and a method for measuring blood pressure capable of correcting an error of blood pressure.

### DISCLOSURE

### Technical Problem

An object of the present invention is to provide a blood pressure meter for measuring blood pressure by using pulse waves, and more particularly, to a blood pressure meter and a method for measuring blood pressure using pulse waves measured at different heights and a height difference between two points where the pulse waves are measured in blood pressure measurement.

### Technical Solution

An aspect of the present invention provides a blood pressure meter comprising: a pulse wave measuring unit for measuring arterial pulse waves; a blood pressure difference calculating unit for calculating a difference between blood pressure values generated by a height difference between two certain points at which the pulse waves are measured; and a blood pressure wave calculating unit for converting the pulse waves measured at the two points into blood pressure waves by using the difference between the blood pressure values. The blood pressure meter according to an aspect of the present invention may be provided as a wearable blood pressure meter, that is, a portable wrist blood pressure meter or finger blood pressure meter that can be worn on a predetermined part of the human body, such as the wrist or the finger. In addition, the present invention can be a blood pressure meter for measuring blood pressure by allowing the finger to be in contact with an optical measuring sensor of a smart phone. The blood pressure wave calculating unit is applied to allow a blood pressure change rate per unit height of pulse waves derived from a difference between the blood pressure values generated by a height difference between the two points to convert the pulse waves into blood pressure waves, wherein the blood pressure change rate may be obtained by [Equation 1] below.

Blood pressure change rate=ΔP/ΔW [Equation 1]

(ΔP represents a difference (blood pressure difference) between blood pressure values generated by a height difference between two certain points where the pulse waves are measured and ΔW represents a difference between pulse waves measured at two certain points)

The blood pressure meter may further include a height difference sensing unit that senses a height difference between the two points where the pulse waves are measured. Of course, the height difference between the two points may also be measured manually, for example, by using a ruler, such as a measuring tape.

The height difference sensing unit may include at least one of an acceleration sensor, a height sensor, a pressure sensor, a differential amplifier, and a gyro sensor. Of course, the height difference sensing unit may use any device capable of measuring a height difference between two certain positions where pulse waves are measured. In addition, an example of the pulse wave measuring unit includes a photoplethysmography, but is not limited thereto, and a sensor capable of measuring pulse waves, for example, a pressure sensor is also possible.

In the blood pressure difference calculating step, a difference between the blood pressure values may be calculated by using Equation 2 below, but is not limited thereto.

ΔP=g×ρ×ΔH [Equation 2]

(g represents the acceleration of gravity, ρ represents the density of blood, and ΔH represents a height difference between two certain points where the pulse waves are measured)

Another aspect of the present invention provides a method for measuring blood pressure comprising: a pulse wave measuring step of measuring arterial pulse waves at two certain points; a blood pressure difference calculating step of calculating a difference between blood pressure values generated by a height difference between two certain points where the pulse waves are measured; and a blood pressure wave calculating step of converting the pulse waves measured at the two points into blood pressure waves by using the difference between the blood pressure values. The blood pressure wave calculating step is applied to allow a blood pressure change rate per unit height of pulse waves derived from a difference between the blood pressure values generated by a height difference between the two points to convert the pulse waves into blood pressure waves, wherein the blood pressure change rate may be obtained by [Equation 1] described above.

The method for measuring the blood pressure may further include a height difference sensing step of sensing a height difference between the two points where the pulse waves are measured, simultaneously with or after the pulse wave measuring step.

In the blood pressure difference calculating step, the difference between the blood pressure values may be calculated by using [Equation 2] described above, but is not limited thereto.

Further, the method for measuring the blood pressure may further include a setting step of measuring an arterial pulse wave and blood pressure at one certain point and setting a reference line of the blood pressure wave, before the pulse wave measuring step.

In the pulse wave measuring step, the pulse waves may be measured on the same part of the body located at different heights with a time interval. Of course, the pulse waves may also be measured by varying heights for different parts of the body.

### Advantageous Effects

According to the present invention, since the blood pressure meter for measuring blood pressure by using pulse waves, more specifically, rapid changes in a blood vessel state which largely influence a blood pressure measurement in addition to a blood flow rate in the blood pressure meter may be reflected in the blood pressure calculation, a correlation between the pulse wave and the blood pressure wave may be reset whenever the blood pressure is measured (resetting of blood pressure conversion reference value), thereby enabling blood pressure to be measured with improved accuracy and greatly improving the accuracy of the blood pressure.

### DESCRIPTION OF DRAWINGS

Features and advantages of the present invention will be more clearly understood with reference to the following drawings to be described below in conjunction with a detailed description of the embodiment(s) of the present invention to be described below, in which:

FIG. 1 is a block diagram illustrating a configuration of a blood pressure meter according to an embodiment of the present invention;

FIG. 2 is a diagram illustrating an example of a method for measuring blood pressure according to an embodiment of the present invention;

FIG. 3 is a graph showing pulse waves measured by an embodiment of the present invention and blood pressure waves obtained by the embodiment of the present invention;

FIG. 4 is a flowchart schematically illustrating a method for measuring blood pressure according to an embodiment of the present invention; and

FIG. 5 is a diagram illustrating an example of a method for measuring blood pressure according to an embodiment of the present invention.

### MODES OF THE INVENTION

Hereinafter, preferred embodiments of the present invention, in which a purpose of the present invention can be realized in detail, will be described with reference to the accompanying drawings. In describing the embodiments, like names and like reference numerals will be used for like configurations and additional description thereof will be omitted.

Terms used in the present application are used only to describe the embodiments of the present invention, and are not intended to limit the present invention. For example, terms including an ordinal number such as “first” and “second” may be used to distinguish components from each other when describing components of the same name, but do not define or limit the number of components.

It should be understood that, when it is described that a component is “connected to” or “accesses” another component, the component may be directly connected to or access the other component, but a connection relation in which other components may be present therebetween, that is, a relation in which other components are indirectly connected may also be included.

In the present application, it should be understood that term “include” or “have” indicates that a feature, a number, a step, an operation, a component, a part or a combination thereof described in the specification is present, but does not exclude a possibility of presence or addition of one or more other features, numbers, steps, operations, components, parts, or combinations.

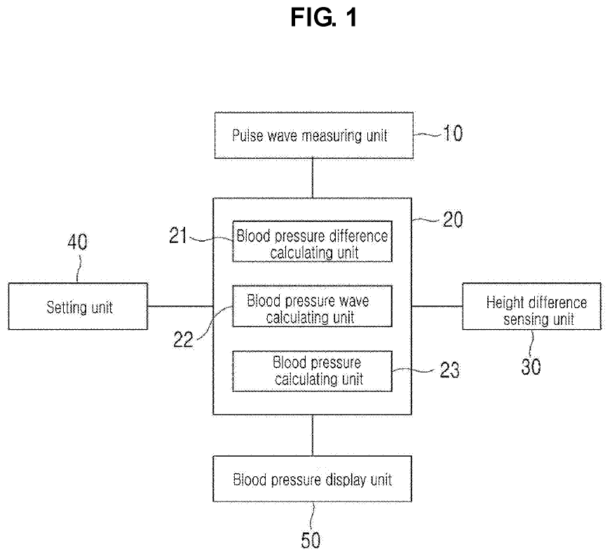

Hereinafter, an embodiment of a blood pressure meter according to the present invention and a method for providing blood pressure using the same will be described with reference to FIGS. 1 to 4.

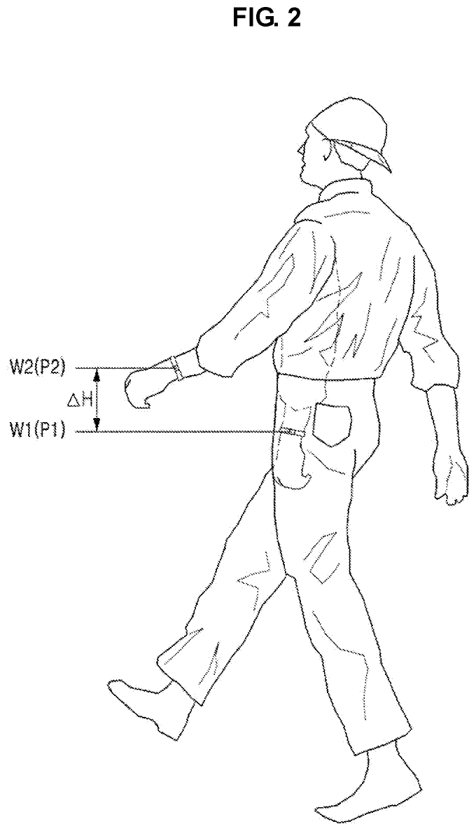

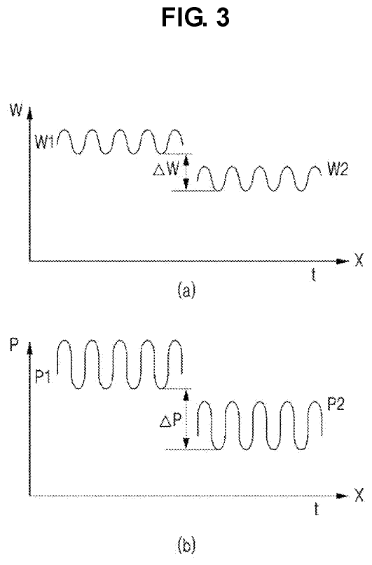

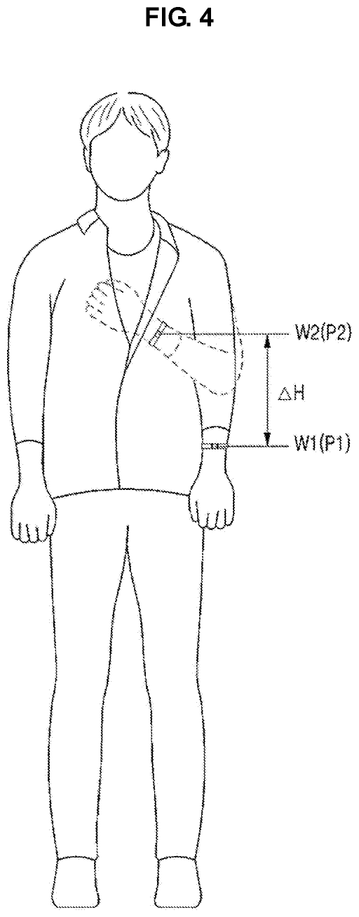

FIG. 1 is a block diagram illustrating a configuration of a blood pressure meter according to an embodiment of the present invention, FIG. 2 is a diagram illustrating an example of a method for measuring blood pressure according to an embodiment of the present invention, FIG. 3 is a graph showing pulse waves measured by an embodiment of the present invention and blood pressure waves obtained by the embodiment of the present invention, and FIG. 4 is a flowchart schematically illustrating a method for measuring blood pressure according to an embodiment of the present invention.

The blood pressure meter according to an embodiment of the present invention is a portable blood pressure meter, and more specifically a wearable blood pressure meter (wearable measuring device of blood pressure).

That is, an aspect of the present invention may be provided as a portable blood pressure meter that is worn on the human body to measure a pulse wave of a part to be measured (target part) and convert an actual value, that is, an actual pulse wave measured at the part to be measured to obtain a blood pressure value.

Referring to FIGS. 1 to 4, the blood pressure meter according to an embodiment of the present invention is configured to include a pulse wave measuring unit 10 for measuring an arterial pulse wave and a blood pressure meter control unit 20 for calculating blood pressure by using the pulse wave.

The blood pressure meter control unit 20 includes a blood pressure difference calculating unit 21 for calculating a difference between blood pressure values, i.e., a blood pressure difference ΔP generated by a height difference ΔH between two certain points where pulse waves W1 and W2 are measured and a blood pressure wave calculating unit 22 for converting the pulse waves W1 and W2 measured at the two points into blood pressure waves P1 and P2 by using the difference ΔP between the blood pressure values.

The blood pressure meter according to an aspect of the present invention may also be provided as a wearable blood pressure meter, that is, a portable wrist blood pressure meter or finger blood pressure meter that can be worn on a predetermined part of the human body, such as the wrist or the finger. In addition, the present invention may also be applied to smart phones, and for example, a blood pressure meter of measuring blood pressure by allowing a finger to be in contact with an optical measuring sensor of the smart phone is possible.

In addition, the blood pressure meter may further include a height difference sensing unit 30 that senses the height difference ΔH between the two points where the pulse waves W1 and W2 are measured. Of course, the height difference ΔH between the two points may also be measured manually, for example, by using a ruler, such as a measuring tape.

The height difference sensing unit 30 may include at least one of an acceleration sensor, a height sensor, a pressure sensor, a differential amplifier, and a gyro sensor. Of course, the height difference sensing unit may use any device capable of measuring a height difference between two certain positions where pulse waves are measured.

The height difference sensing unit 30 is a configuration for sensing height changes and senses a height of the target part when the pulse wave is measured at the target part (a body part where the pulse wave is measured) by the pulse wave measuring unit 10 and detects a height difference between positions where the pulse waves are measured.

In other words, the height difference sensing unit 30 is a configuration of sensing height changes in the blood pressure meter worn on the target part. When a person (user) wearing the blood pressure meter is walking, a body organ (arm) in which the target part (e.g., the wrist) is located moves back and forth according to a user's walking pattern. More specifically, when the user shakes the arm back and forth while walking, a height of the blood pressure meter worn on the target part (wrist) is changed, and the height difference sensing unit 30 senses the height of the blood pressure meter while the arm moves as described above.

In addition, an example of the pulse wave measuring unit 10 includes a photoplethysmography, but is not limited thereto, and a sensor capable of measuring pulse waves, for example, a pressure sensor is also possible.

The blood pressure difference calculating unit 21 may calculate a difference ΔP between the blood pressure values, that is, a blood pressure difference by using the following [Equation], but is not limited thereto.

ΔP=g×ρ×ΔH [Equation]

(ΔP represents a difference between blood pressure values generated by a height difference between two certain points where the pulse waves are measured, g represents the acceleration of gravity, ρ represents the density of blood, and ΔH represents a height difference between two certain points where the pulse waves are measured)

The density ρ of the blood may be an actually measured value or a predetermined average value.

The blood pressure meter according to an embodiment of the present invention is a blood pressure meter for measuring pulse waves and calculating blood pressure by using the measured pulse waves. As illustrated in FIG. 2, the blood pressure meter converts the pulse waves into blood pressure waves (waveforms of blood pressure) by measuring continuously arterial pulse waves, for example, optical arterial pulse waves W1 and W2 and heights at two certain points having a height difference when the user measures the blood pressure while walking and displays (outputs) the user's blood pressure. The optical arterial pulse wave described above refers to an arterial pulse wave measured by the photoplethysmography.

More specifically, the blood pressure meter according to an embodiment of the present invention measures continuously arterial pulse waves, for example, optical arterial pulse waves W1 and W2 and heights at two certain points, converts pulse waves (optical arterial pulse waves) W1 and W2 into blood pressure waves P1 and P2 as illustrated in FIG. 3 by setting a blood pressure difference ΔP by the height difference ΔH between the two points as a blood pressure conversion value, and calculates and displays the blood pressure. Therefore, the blood pressure conversion reference value may be reset whenever the blood pressure is measured to reflect a blood vessel state for calculating the blood pressure. The blood vessel state at the time of measuring the blood pressure may be reflected by applying a blood pressure difference by the height difference ΔH to pulse wave-blood pressure wave conversion so that a difference between waveforms when converting the two pulse waves (optical arterial pulse waves; W1 and W2) into the blood pressure waves P1 and P2, which is a difference ΔW between the pulse waves, becomes the blood pressure difference ΔP.

In addition, in the blood pressure meter, a relation between the pulse wave and the blood pressure is set through a setting process, i.e., a calibration process. To this end, the blood pressure meter includes a setting unit 40, and the setting unit 40 measures an arterial pulse wave and blood pressure at one certain point, and sets a reference line of the blood pressure wave by the pulse wave and THE blood pressure measured at this time. The reference line X is set by substituting the measured blood pressure value into the measured pulse wave, and since the calibration process itself is generally known in the tonometric blood pressure meter, additional description thereof will be omitted.

In addition, the blood pressure meter control unit 20 further includes a blood pressure calculating unit 23 for calculating blood pressure from the pulse waves, and the blood pressure value obtained by the blood pressure calculating unit 23 is displayed on the blood pressure display unit 50. The blood pressure calculating unit 23 calculates a blood pressure value at a heart height (a blood pressure value measured when the target part is located at the heart height) from the blood pressure wave.

Since the technique for calculating the blood pressure from the pulse waves and the technique for correcting the blood pressure value measured at a certain point to a blood pressure value at a heart height are already known, additional description thereof will be omitted.

Referring to FIG. 3, optical arterial pulse waves W1 and W2 and heights are measured at a bottom dead point (while the arm is placed down, i.e., hangs in a gravity direction) of a wrist blood pressure meter M which the user moves while wearing on the wrist during walking and another certain point located on a moving trace of the wrist, respectively, and the optical arterial pulse waves W1 and W2 are converted into the blood pressure waves P1 and P2 by applying a blood pressure change rate (ΔP/ΔW) per unit height of the optical arterial pulse waves at a reference line X of the pulse wave measured during setting (calibration) and the blood pressure wave set by the blood pressure value and a blood pressure difference (ΔP=g×ρ×ΔH) of the two optical arterial pulse waves W1 and W2 by the height difference ΔH. In other words, the difference ΔP between the blood pressure values generated by the height difference at the two points, i.e., the blood pressure change rate (ΔP/ΔW) per unit height of the pulse wave derived from the blood pressure difference is applied to convert the pulse wave into the blood pressure wave, and as a result, it can be seen that the pulse wave-blood pressure wave conversion by the blood pressure wave calculating unit 22 is performed. In the converted blood pressure waves P1 and P2, a peak is a maximum blood pressure and the valley is a minimum blood pressure.

Accordingly, a method for measuring blood pressure according to an embodiment of the present invention includes a pulse wave measuring step of measuring arterial pulse waves at two certain points; a blood pressure difference calculating step of calculating a difference between blood pressure values generated by a height difference between two certain points where the pulse waves are measured; and a blood pressure wave calculating step of converting the pulse waves measured at the two points into blood pressure waves by using the difference between the blood pressure values.

The method for measuring the blood pressure may further include a height difference sensing step of sensing a height difference between the two points where the pulse waves are measured, simultaneously with or after the pulse wave measuring step, and in the blood pressure difference calculating step, a difference (blood pressure difference; ΔP) between the blood pressure values may be calculated by using Equation (ΔP=g×ρ×ΔH) described above.

Further, the method for measuring the blood pressure may further include a setting step of measuring an arterial pulse wave and blood pressure at one certain point and setting a reference line of the blood pressure wave, before the pulse wave measuring step, for example, in a blood pressure meter initializing step.

In the pulse wave measuring step, like an example illustrated in FIG. 3, the pulse waves may be measured on the same part of the body (the same target part) at different heights with a time interval.

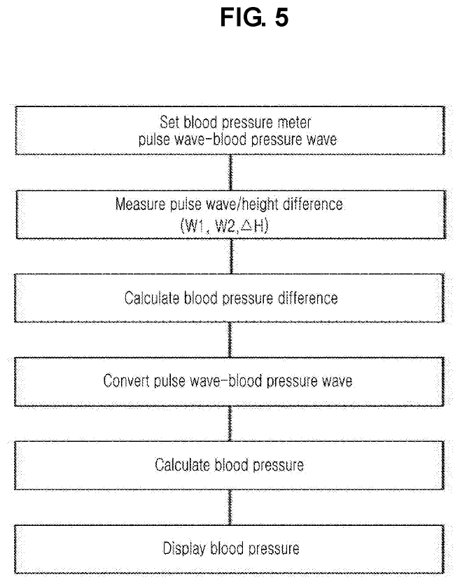

FIG. 5 is a diagram illustrating another example of the method for measuring the blood pressure. In the method, optical arterial pulse waves and heights are measured at a bottom dead point (while the arm hangs in a gravity direction) of the wrist while the user is standing or sitting and another point other than the bottom dead point, for example, a wrist (target part) while being located at a heart height, respectively, the pulse waves are converted into the blood pressure waves by using the height difference between the two points and the blood pressure difference according to the height difference, and the blood pressure may be calculated from the blood pressure wave.

Of course, the blood pressure meter may measure pulse waves by varying heights for different parts of the body and calculate the blood pressure by using the measured pulse waves. In other words, the blood pressure meter may measure pulse waves simultaneously at different heights by varying target parts (parts where the pulse waves are measured) and calculate the blood pressure based on the value.

The aforementioned description of the present invention is to be illustrative, and it will be understood to those skilled in the art that the technical spirit or required features of the present invention can be easily modified in other detailed forms without changing. Therefore, it should be understood that the above-described embodiments are illustrative in all aspects and do not limit the present disclosure. For example, respective constituent elements described as single types can be distributed and implemented, and similarly, constituent elements described to be distributed can also be implemented in a coupled form.

The scope of the present invention is represented by claims to be described below rather than the detailed description, and it is to be interpreted that the meaning and scope of the claims and all the changes or modified forms derived from the equivalents thereof come within the scope of the present invention.

1. A blood pressure meter comprising:
a pulse wave measuring unit for measuring arterial pulse waves;
a blood pressure difference calculating unit for calculating a difference between blood pressure values generated by a height difference between two certain points at which the pulse waves are measured; and
a blood pressure wave calculating unit for converting the pulse waves measured at the two points into blood pressure waves by using the difference between the blood pressure values,
wherein the blood pressure wave calculating unit applies a blood pressure change rate per unit height of pulse waves derived from a difference between the blood pressure values generated by a height difference between the two points to convert the pulse waves into blood pressure waves, wherein the blood pressure change rate is obtained by [Equation 1] below.
Blood pressure change rate=ΔP/ΔW [Equation 1]
(ΔP represents a difference (blood pressure difference) between blood pressure values generated by a height difference between two certain points where the pulse waves are measured and ΔW represents a difference between pulse waves measured at two certain points)

2. The blood pressure meter of claim 1, further comprising:
a height difference sensing unit that senses a height difference between the two points where the pulse waves are measured.

3. The blood pressure meter of claim 2, wherein the height difference sensing unit includes at least one of an acceleration sensor, a height sensor, a pressure sensor, a differential amplifier, and a gyro sensor.

4. The blood pressure meter of claim 1, wherein the pulse wave measuring unit includes a photoplethysmography.

5. The blood pressure meter of claim 1, wherein the blood pressure difference calculating unit calculates the difference between the blood pressure values by using [Equation 2] below.
ΔP=g×ρ×ΔH [Equation 2]
(g represents the acceleration of gravity, ρ represents the density of blood, and ΔH represents a height difference between two certain points where the pulse waves are measured)

6. A method for measuring blood pressure comprising:
a pulse wave measuring step of measuring arterial pulse waves at two certain points;
a blood pressure difference calculating step of calculating a difference between blood pressure values generated by a height difference between two certain points where the pulse waves are measured; and
blood pressure wave calculating step of converting the pulse waves measured at the two points into blood pressure waves by using the difference between the blood pressure values,
wherein the blood pressure wave calculating step is applied to allow a blood pressure change rate per unit height of pulse waves derived from a difference between the blood pressure values generated by a height difference between the two points to convert the pulse waves into blood pressure waves, wherein the blood pressure change rate is obtained by [Equation 1] below.
Blood pressure change rate=ΔP/ΔW [Equation 1]
(ΔP represents a difference (blood pressure difference) between blood pressure values generated by a height difference between two certain points where the pulse waves are measured and ΔW represents a difference between pulse waves measured at two certain points)

7. The method for measuring blood pressure of claim 6, further comprising:
a height difference sensing step of sensing a height difference between the two points where the pulse waves are measured, simultaneously with or after the pulse wave measuring step.

8. The method for measuring blood pressure of claim 7, wherein in the blood pressure difference calculating step, a difference between the blood pressure values is calculated by using [Equation 2] below.
ΔP=g×ρ×ΔH [Equation 2]
(g represents the acceleration of gravity, ρ represents the density of blood, and ΔH represents a height difference between two certain points where the pulse waves are measured)

9. The method for measuring blood pressure of claim 6, further comprising:
a setting step of measuring an arterial pulse wave and blood pressure at one certain point and setting a reference line of the blood pressure wave, before the pulse wave measuring step.

10. The method for measuring blood pressure of claim 6, wherein in the pulse wave measuring step, the pulse waves are measured on the same part of the body located at different heights with a time interval in the same part of the body.

11. The method for measuring blood pressure of claim 7, further comprising:
a setting step of measuring an arterial pulse wave and blood pressure at one certain point and setting a reference line of the blood pressure wave, before the pulse wave measuring step.

12. The method for measuring blood pressure of claim 8, further comprising:
a setting step of measuring an arterial pulse wave and blood pressure at one certain point and setting a reference line of the blood pressure wave, before the pulse wave measuring step.

13. The method for measuring blood pressure of claim 7, wherein in the pulse wave measuring step, the pulse waves are measured on the same part of the body located at different heights with a time interval in the same part of the body.

14. The method for measuring blood pressure of claim 8, wherein in the pulse wave measuring step, the pulse waves are measured on the same part of the body located at different heights with a time interval in the same part of the body.
